(19) United States
(12) Patent Application Publication
(10) Pub. No.: US 2007/0249694 A1
(43) Pub. Date: Oct. 25, 2007

(54) Metaxalone formulations and methods for the preparation thereof

(76) Inventors: Caroline Abrutzky Azaria, Kfar Saba (IL); Gershon Kolatkar, Petah Tiqva (IL); Mira Kopel, Katzir (IL); Fanny Leska, Herzliya (IL); Ilan Zalit, Rosh Ha Ayin (IL)

Correspondence Address: KENYON & KENYON LLP, ONE BROADWAY, NEW YORK, NY, 10004, US

(21) Appl. No.: 11/648,309

(22) Filed: Dec. 29, 2006

Related U.S. Application Data
(60) Provisional application No. 60/755,577, filed on Dec. 29, 2005.

(51) Int. Cl. A61K 31/42

(52) U.S. Cl. 514/376

(57) ABSTRACT

The invention provides pharmaceutical metaxalone compositions having improved bioavailability, food effect, and/or relative food effect, as well as methods of making such metaxalone compositions.

### RELATED APPLICATIONS

The present application claims benefit of U.S. Provisional Application No. 60/755,577, filed Dec. 29, 2005, the contents of which are incorporated in their entirety by reference herein.

### BACKGROUND OF THE INVENTION

Metaxalone is marketed in the United States under the brand name SKELAXIN® by Elan Pharmaceuticals, Inc., in 400 mg and 800 mg tablets. SKELAXIN® is indicated as an adjunct to rest, physical therapy, and other measures for the relief of discomforts associated with acute, painful musculoskeletal conditions. The mode of action of this drug has not been clearly identified, but may be related to its sedative properties. Reportedly, metaxalone does not directly relax tense skeletal muscles in humans.

Metaxalone is reportedly a tasteless, odorless, white crystalline powder that melts without decomposition at 121.5° to 123° C. The compound has the chemical name 5-[(3,4-dimethylphenoxy)methyl]-2-oxazolidinone, and the following chemical formula:

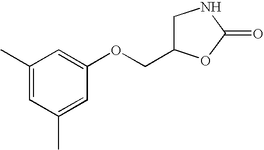

U.S. Pat. Nos. 6,407,128 and 6,683,102 disclose a method of increasing the oral bioavailability of metaxalone to a patient receiving metaxalone therapy. In the disclosed method, a therapeutically effective amount of metaxalone in a pharmaceutical composition is administered to the patient with food. Significant differences in the values of both Cₘₐₓ and AUC are reported in the '128 and '102 patents for the commercially available metaxalone composition, SKELAXIN®, between the fasting and fed states in healthy humans. Both the rate and extent of absorption of the commercial product are reportedly significantly higher for fed patients than for fasting patients. As defined in the '128 and '102 patents, the “fed” state refers to a period between 30 minutes before and 2 hours after a meal, and the “fast” state refers to a period at least 2 hours after eating. The difference between the bioavailability in the “fed” and “fast” states is the “food effect,” and can interfere with patient compliance.

PCT Application Publication WO 2004/019937 discloses metaxalone pharmaceutical compositions having reportedly enhanced bioavailability, compared to the metaxalone of commercially available SKELAXIN® tablets. The PCT Publication defines the bioavailability as the rate and extent of absorption of the metaxalone. Specific particle size distribution characteristics are disclosed in the examples that reportedly enhance bioavailability, which is reportedly independent of whether the metaxalone is administered with food or with fasting. The metaxalone in the disclosed compositions is a “pharmaceutically acceptable solubility-improved form” that includes “micronized” metaxalone, a salt form of metaxalone, a “high-energy crystalline” form of metaxalone, and amorphous metaxalone. Reportedly, “micronized” metaxalone may be obtained either by crystallization of metaxalone or spray drying or by the use of conventional milling techniques. The disclosed “pharmaceutically acceptable solubility-improved form” of metaxalone provides a reduced particle size distribution that reportedly enhances the bioavailability relative to the commercially available metaxalone product, SKELAXIN®.

U.S. Patent Application Publication No. 2005/0063913 discloses nanoparticulate compositions comprising metaxalone or a salt of metaxalone, having an effective average particle size of less than about 2 μm, and at least one surface stabilizer. An increased Cₘₐₓ, i.e., the maximum blood/serum concentration, a reduced Tₘₐₓ, i.e., the time to Cₘₐₓ, and an increased AUC, i.e., the area under the curve, a measure of the total patient exposure to the drug, are reported. An insignificant difference in the bioequivalency of the disclosed nanoparticulate compositions of metaxalone under fed and fasting conditions upon administration to healthy mammals is also reported.

PCT application publication WO 2005/087204 discloses metaxalone tablet formulations, reportedly having equivalent or improved dissolution and/or oral bioavailability relative to the commercial product, where “drug dissolution rate” is defined as the amount of drug dissolved within a given time period, and “oral bioavailability” is defined as the measure of the rate and extent of drug absorption in healthy human volunteers, as expressed by Cₘₐₓ. Two of the disclosed metaxalone tablet formulations reportedly show higher bioavailability when administered to healthy patients in the fasting state. One other disclosed formulation comprises ammonium alginate and sodium alginate, and is reportedly bioequivalent to SKELAXIN®. Due to the “maximally enhanced bioavailability properties” of the disclosed metaxalone formulations under fasting conditions, no oral bioavailability food effect is reportedly expected.

A disadvantage in the use of either of the alginates is that such excipients are hygroscopic. As a result, special care must be taken during preparation of formulations comprising sodium or ammonium alginate to maintain a low relative humidity and cool temperatures. In addition, because alginates are derived from naturally occurring materials, the physical properties of the excipient can vary from batch to batch. A difference in the physical properties can cause variations in the pharmacokinetic properties between batches of a drug product.

Metaxalone compositions having improved bioavailability, food effect, and/or relative food effect, as well as a method that provides a process for adjusting the food effect and/or the relative food effect of a metaxalone formulation would be advantageous. The present invention provides such a compositions and such a method.

### SUMMARY OF THE INVENTION

The present invention is directed to pharmaceutical compositions, comprising more than 400 mg of metaxalone, at least one binder, and at least one disintegrant, wherein the weight ratio between the binder and metaxalone is in the range of about 1 percent to about 10 percent, and the weight ratio between the disintegrant and metaxalone is in the range of about 0.5 percent to about 10 percent, and to methods of preparing such pharmaceutical metaxalone compositions, having controlled bioavailability and controlled relative food effect. The methods of the invention comprise the steps of:

(a) preparing a pharmaceutical composition comprising more than 400 mg of metaxalone together with at least one binder and at least one disintegrant;

(b) calculating the food effect and/or the relative food effect;

(c) adjusting the binding capacity of the binder and the disintegration capacity of the disintegrant to achieve a desired food effect and/or relative food effect; and

(d) preparing a pharmaceutical composition, comprising more than 400 mg of metaxalone together with at least one binder and at least one disintegrant, having the adjusted binding capacity and disintegration capacity, wherein the weight ratio between the binder and metaxalone is in the range of about 1 percent to about 10 percent, and the weight ratio between the disintegrant and metaxalone is in the range of about 0.5 percent to about 10 percent; and

(e) determining the food effect and/or the relative food effect of the prepared metaxalone composition.

### DETAILED DESCRIPTION OF THE INVENTION

As used herein, the term “metaxalone” refers to the compound 5-[(3,4-dimethylphenoxy)methyl]-2-oxazolidinone, having the following chemical structure:

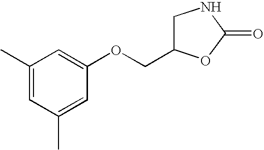

As used herein, for metaxalone, the term “Cₘₐₓ” refers to maximum concentration of metaxalone in blood or serum following the ingestion of a dose of metaxalone. As used herein, for metaxalone, the term “Tₘₐₓ” refers to the time following ingestion required for the level of metaxalone in blood or serum to reach Cₘₐₓ. As used herein, for metaxalone, the term “AUC” refers to the area under the curve of a plot of the blood/serum concentration of metaxalone against time from the time metaxalone is ingested until the concentration is below the detection limit. “AUC” is a measure of the total patient exposure to metaxalone.

As used herein, the “binder capacity” or “binding capacity” of a binder are a function of the amount of binder and the binding capability of a binder, where the binding capability is the binding strength per unit weight of a binder. Therefore, the binding capacity of a binder used in the compositions of the invention can be adjusted by increasing the amount of a particular binder, by changing the type of binder to one with a different binding capability, or by adding additional binder of a different type to the initial binder. For example, where polyvinylpyrrolidone K-30, which has a molecular weight of about 40,000, is the initial binder, additional polyvinylpyrrolidone K-30 can be added, or the polyvinylpyrrolidone K-30 can be replaced with polyvinylpyrrolidone K-90, which has a molecular weight of about 360,000, or polyvinylpyrrolidone K-90 can be added to the polyvinylpyrrolidone K-30, any of which will increase the binding capacity.

Similarly, the “disintegrant capacity” or “disintegration capacity” is a function of the amount of disintegrant and the disintegration capability of the disintegrant, where the disintegration capability is defined as the disintegration strength per unit weight of the disintegrant. Therefore, the disintegration capacity of a disintegrant used in the compositions of the invention can be increased by increasing the amount of a particular disintegrant, by changing the type of disintegrant to one with a different, higher disintegration capability, or by adding additional disintegrant of a different type to the initial disintegrant, and the disintegration capacity of a disintegrant used in the compositions of the invention can be decreased by decreasing the amount of a particular disintegrant, or by changing at least a portion of the disintegrant to a type of disintegrant with a different, lower disintegration capability.

As used herein, the term “Cₘₐₓ ratio” refers to the ratio of the Cₘₐₓ of a composition of the invention to the Cₘₐₓ of the commercial product, e.g., SKELAXIN®. That is, the Cₘₐₓ ratio is defined as
Cₘₐₓ ratio=Cₘₐₓ/Cₘₐₓᵢ,
where Cₘₐₓ is the Cₘₐₓ of a composition of the invention, and Cₘₐₓᵢ is the Cₘₐₓ of the innovator's product, i.e., the present commercial product. In addition, as used herein, the food effect of a metaxalone composition is the ratio of the Cₘₐₓ under fed conditions, i.e., fed Cₘₐₓ, to the Cₘₐₓ under fasted conditions, i.e., fast Cₘₐₓ. That is, the food effect, F.E., is defined as
F.E.=Cₘₐₓ fed/Cₘₐₓ fast.

Similarly, as used herein, the term “relative food effect” refers to ratio of the Cₘₐₓ ratio under fed conditions, i.e., the fed Cₘₐₓ ratio, to the Cₘₐₓ ratio under fasted conditions, i.e., the fast Cₘₐₓ ratio. That is the relative food effect, R.F.E., is defined as
R.F.E.=fed Cₘₐₓ ratio/fast Cₘₐₓ ratio.
As will be recognized by one of ordinary skill in the art, the relative food effect is the ratio of the food effect of a metaxalone composition of the invention to the food effect of the commercial metaxalone composition. That is, as
R.F.E.=fed Cₘₐₓ ratio/fast Cₘₐₓ ratio, and
Cₘₐₓ ratio=(Cₘₐₓ/Cₘₐₓᵢ),
R.F.E.=(Cₘₐₓ fed/Cₘₐₓᵢ fed)/(Cₘₐₓ fast/Cₘₐₓᵢ fast).
Therefore,
R.F.E.=(Cₘₐₓ fed/Cₘₐₓ fast)/(Cₘₐₓᵢ fed/Cₘₐₓᵢ fast), or R.F.E.=F.E./F.E.ᵢ,
where F.E. is the food effect of the metaxalone composition of the invention, and F.E.ᵢ is the food effect of the commercial metaxalone composition.

Although a reduction in particle size distribution has been reported in the prior art as a means to improve the bioavailability of metaxalone, the need to reduce the particle size is a disadvantage. Reducing the particle size of the metaxalone requires extra steps in the manufacturing process, increasing costs, and will result in the loss of a certain amount of metaxalone during the particle size reduction process, such that the ultimate yield of the final product is also reduced, further increasing production costs.

The present invention encompasses metaxalone formulations having improved bioavailability, controlled bioavailability, and/or a predetermined food effect, which may be minimized, maximized, or similar to that of prior art, commercially available metaxalone, as desired, without the need to reduce the particle size of metaxalone in the formulation.

The present invention provides a pharmaceutical composition comprising more than 400 mg of metaxalone with at least one binder and at least one disintegrant, wherein the weight ratio between the binder and metaxalone is in the range of about 1 percent to about 10 percent, and the weight ratio between the disintegrant and metaxalone is in the range of about 0.5 percent to about 10 percent. The binding capacity and the disintegration capacity of the formulation are adjusted to control the bioavailability of the metaxalone formulations of the invention.

Preferably, the pharmaceutical composition has a “relative food effect” of about 0.6 to about 1.2. Preferably, the “food effect” is of about 1 to about 1.8.

Preferably, the binder comprises a polyvinylpyrrolidone compound, such as Povidone K-30 or Povidone K-90. More preferably, the binder comprises Povidone K-90.

Preferably, the disintegrant comprises a molecule having at least one carboxylic group, such as sodium carboxymethylcellulose, croscarmellose sodium, and sodium starch glycolate. More preferably, the disintegrant is at least one of sodium starch glycolate and an internally cross-linked form of sodium carboxymethylcellulose (NaCMC), such as croscarmellose sodium, where the cross-linking renders the material essentially water insoluble, such as those available commercially as AC-DI-SOL® from FMC BioPolymer. Most preferably, the binder comprises sodium starch glycolate. Preferably, the disintegrant is sodium starch glycolate and the binder is a polyvinylpyrrolidone. More preferably, the disintegrant is sodium starch glycolate and the binder is a polyvinylpyrrolidone having an average molecular weight of about 360,000, such as Povidone K-90.

Preferably, the weight ratio between the disintegrant and metaxalone is in the range of about 0.5 percent to about 3 percent.

Preferably, the weight ratio between the binder and metaxalone is in the range of about 3 percent to about 9 percent.

The invention also encompasses methods for controlling at least one of the bioavailability, the food effect, and the relative food effect of a metaxalone composition by controlling the binding capacity and the disintegration capacity of the composition. In the formulations of the invention, the binding capacity is controlled by adjusting the amount of binder used and the specific binding capability of the binder, which is defined as the binding strength per unit weight binder. Similarly, the disintegration capacity is controlled by adjusting the amount of disintegrant used and the specific disintegration capability of the disintegrant, which is defined as the disintegration strength per unit weight of the disintegrant.

The bioavailability and the food effect in the metaxalone formulations of the invention are controlled by the ratio of the binding and disintegrant capacities and the ratio of each of those capacities and the amount of metaxalone in the formulation. A greater disintegration capacity at a given binding capacity provides an increase in both Cₘₐₓ and AUC values under fasting and fed conditions. However, when increasing both binding capacity and disintegration capacity, the effect on the Cₘₐₓ and AUC values is substantially smaller when the metaxalone is taken with food.

Preferably, the invention reduces the food effect by increasing the disintegration capacity of a metaxalone formulation. More preferably, the food effect is reduced with the formulations and methods of the invention by increasing both the binding capacity and the disintegration capacity of the metaxalone formulation. Preferred metaxalone formulations in accordance with the invention can be administered both under fed and fasting conditions, while maintaining high values of Cₘₐₓ and AUC.

The present invention provides a method for producing a pharmaceutical composition comprising more than 400 mg of metaxalone with at least one binder and at least one disintegrant, wherein the weight ratio between the binder and metaxalone is in the range of about 1 percent to about 10 percent, and the weight ratio between the disintegrant and metaxalone is in the range of about 0.5 percent to about 10 percent, having controlled bioavailability and controlled relative food effect as described above. The method comprises:

(a) preparing a pharmaceutical composition comprising more than 400 mg of metaxalone together with at least one binder and at least one disintegrant;

(b) calculating the relative food effect;

(c) adjusting the binding capacity of the binder and the disintegration capacity of the disintegrant to achieve the desired relative food effect; and

(d) preparing a pharmaceutical composition, comprising more than 400 mg of metaxalone together with at least one binder and at least one disintegrant, having the adjusted binding capacity and disintegration capacity, wherein the weight ratio between the binder and metaxalone is in the range of about 1 percent to about 10 percent, and the weight ratio between the disintegrant and metaxalone is in the range of about 0.5 percent to about 10 percent; and

(e) determining the food effect and/or the relative food effect of the prepared metaxalone composition.

Preferably, increasing or decreasing the amount or the strength of the binder and the disintegrant increases or decreases the metaxalone bioavailability for fasted conditions. Preferably, the metaxalone bioavailability for fed conditions is substantially unchanged.

When the food effect and/or relative food effect of the metaxalone composition is greater than the desired food effect and/or relative food effect, the amount and/or the strength of the binder and the disintegrant is increased, and step (d), as modified above, and step (e) are repeated until a pharmaceutical composition comprising more than 400 mg of metaxalone, having a food effect and/or relative food effect value about that of the desired food effect and/or relative food effect, is obtained. Similarly, when the food effect and/or relative food effect of the metaxalone composition is less than the desired food effect and/or relative food effect, the amount and/or the strength of the binder and the disintegrant is decreased, and step (d), as modified above, and step (e) are repeated until a pharmaceutical composition comprising more than 400 mg of metaxalone, having a food effect and/or relative food effect value about that of the desired food effect and/or relative food effect, is obtained. Preferably, the desired food effect is about 1 to about 1.8 and/or the desired relative food effect is about 0.6 to about 1.2.

Preferably, increasing or decreasing the amount and/or the strength of the binder and the disintegrant increases the metaxalone bioavailability for fasted conditions, and, more preferably, the metaxalone bioavailability for fed conditions are substantially unchanged. The amount and/or the strength of the binder and the disintegrant is preferably increased sufficiently to provide a food effect of about 1 and/or a relative food effect of about 0.6. The invention is also directed to pharmaceutical compositions comprising more than 400 mg of metaxalone prepared by the methods of the invention.

Although the food effect and/or the relative food effect of compositions in accordance with the invention can be adjusted by adjusting just one of the binding capacity or the disintegration capacity of a composition, it is preferred that the binding capacity and the disintegration capacity of the composition are increased or decreased together. For example, when the food effect and/or relative food effect of a metaxalone composition is greater than the desired food effect and/or relative food effect, the amount and/or the strength of the binder and/or the disintegrant is increased.

By adjusting the amount of at least one of the binder and the disintegrant in a formulation of the invention, the Cₘₐₓ ratios, i.e., the ratio of the Cₘₐₓ of the formulation to that of commercial metaxalone, SKELAXIN®, can be adjusted from 50 percent and 301 percent, for the fasting and fed conditions, respectively, to 177 percent and 125 percent, respectively. As a result of the change in the formulation of the invention, the food effect, as determined by the Cₘₐₓ, is adjusted from 0.17 to 1.4, i.e., by a factor of more than 8. Similarly, the food effect, as determined by the AUC, is adjusted from 0.57 to 1.15, a factor of almost 2. Moreover, in the presence of a relatively large amount of binder, the effect on fasting is greater than those on fed for both Cₘₐₓ and AUC, where the values change by 107 percent and 42 percent, respectively, is greater than the effect on fed with 20 percent and 18 percent, respectively. The results clearly show the effect of changes in the formulation on the absorption rate and on the gap between fast and fed absorption rates, parameters which play an important role in the effectiveness and safety of the drug.

### EXAMPLES

### Examples 1, 2, 3

The experimental batches used in Example 1, Example 2, and Example 3 were manufactured by wet granulation. The batches were manufactured using a high shear mixer and fluidized bed drier (Part 1). The extra-granular excipients were added to the milled granulate and mixed in a blender (Part 2). Tablet cores were compressed. In Example 1, a relatively low level of binder and a low level of disintegrant were used. In Example 2, a stronger binder, PVP K-90 (polyvinylpyrrolidone K-90); which is poly[1-(2-oxo-1-pyrrolidinyl)ethylene], having an average molecular weight of about 360,000, rather than the PVP K-30 (polyvinylpyrrolidone K-30); which is poly[1-(2-oxo-1-pyrrolidinyl)ethylene], having an average molecular weight of about 40,000, used in Example 1, was used with a similar amount of disintegrant. In Example 3, a high level of the strong binder and a relatively high level of disintegrant were used. With the exception of the tablets of example 1, which were tested only under fasting conditions, the formulations were tested under fast and fed conditions.

The results demonstrate the effect of the binder, the disintegrant, and the balance between the binder and the disintegrant on the fasting/fed absorption rates. Using a low level of the weak binder in Example 1 resulted in a high fasting Cₘₐₓ and AUC ratios. Replacing the binder of Example 1 with a stronger binder, while maintaining the content of disintegrant, resulted in a significant decrease in the fasting Cₘₐₓ and AUC ratios (fasting Cₘₐₓ from 263 percent to 50 percent), while fed results remained very high (Cₘₐₓ ratio fed 301 percent), resulting in a very large gap between the fast and fed results (Cₘₐₓ Fast/Cₘₐₓ Fed=0.17).

Increasing both binder and disintegrant (example 3) resulted in a marked conversion of the ratio between fast and fed results (Cₘₐₓ fasting 177 percent; fed 125 percent; Cₘₐₓ Fast/Cₘₐₓ Fed=1.4).

The relative food effect is defined as Cₘₐₓ ratio Fed/Cₘₐₓ ratio Fast. A bioequivalent formulation to SKELAXIN® would have a relative food effect of 1. If the food effect is lower than one, the formulation of the invention will have a lower food effect than the SKELAXIN® product. A formulation with a relative food effect higher than 1 will exhibit a higher food effect than SKELAXIN®.

TABLE 1

| | Example 1 | Example 2 | Example 3 |
|---|---|---|---|
| | K-27605 | K-28499 | K-30592/2 |
| Part 1 | | | |
| Metaxalone | 400 | 400 | 400 |
| Sodium Starch Glycolate NF | 10 | 5 | 7.5 |
| Povidone USP (PVP K-30) | 13 | | |
| Povidone USP (PVP K-90) | | 10 | 25 |
| Alcohol 95% | | | |
| Part 2 | | | |
| Microcrystalline Cellulose | 53 | 66 | 46 |
| (Avichel PH102) | | | |
| Sodium Starch Glycolate NF | | 5 | 10 |
| Magnesium Stearate NF | 4 | 4 | 4 |
| Color FDC Red#40 Al. Lake | 0.005 | | |
| Color FDC Blue No. 2 | 0.05 | | |
| Hardness | 11scu | 12scu | 12-14scu |
| Fast Cₘₐₓ ratio % | 263 | 50 | 177 |
| Fast AUC ratio % | 150 | 71 | 124 |
| Fed Cₘₐₓ ratio % | NA | 301 | 125 |
| Fed AUC ratio % | NA | 125 | 108 |
| Relative food effect | NA | 0.17 | 1.4 |

### Example 4, 5, 6

The tablets of examples 4, 5, and 6 were manufactured by wet granulation. The batches were manufactured using a high shear mixer and fluidized bed drier (Part 1). The extragranular excipients were added to the milled granulate and mixed in a blender (Part 2). Tablet cores were compressed. The particle size of metaxalone used in these examples was as follows:

d(0.1): 10.5 microns

d(0.5): 52.2 microns

d(0.9): 185.7 microns

The formulations of examples 4-6 are examples of using a high level of strong binder, while changing the content of disintegrant (from high to low). The formulations were tested under fasting and fed conditions.

The results obtained clearly demonstrate an aspect of a preferred embodiment of the invention: using high capacity of binder and changing the disintegration capacity enables good control of the ratio of the fed versus fasting pharmacokinetic value (see Table 2.) Furthermore, the results show that, at those conditions, the effects on fasting (107 percent and 42 percent, for Cₘₐₓ and AUC respectively, comparing examples 6 and 4), are greater than those on fed (20 percent and 18 percent, respectively).

An additional way to demonstrate the reduced food effect of formulation 4 when compared to formulation 6 and innovator (SKELAXIN®) is by comparison of the absolute pK values between the fed and fast studies. It should be mentioned that this way of demonstrating the different food effect of the various formulations is less preferred since the fed and fast studies were performed separately. Table 3 shows that when comparing the absolute pK values the Cₘₐₓ food effect of formulation 4 is 70 percent (calculated as ratio between Cₘₐₓ fast and Cₘₐₓ fed), while for formulation 6 and for SKELAXIN®, higher Cₘₐₓ food effect was observed (50 percent and 50-60 percent respectively). For the AUC less significant differences were observed between the different formulations.

TABLE 2

| | Example 4 | Example 5 | Example 6 |
|---|---|---|---|
| Part 1 | | | |
| Metaxalone | 800 | 800 | 800 |
| Sodium Starch Glycolate NF | 15 | 15 | 13.8 |
| Povidone USP (PVP K-90) | 50 | 50 | 50 |
| Alcohol 95% | | | |
| Part 2 | | | |
| Microcrystalline Cellulose | 104 | 111.5 | 114 |
| (Avichel PH102) | | | |
| Sodium Starch Glycolate NF | 10 | 2.5 | 0 |
| Magnesium Stearate NF | 8 | 8 | 8 |
| Color FDC Red#40 Al. Lake | 0.1 | 0.1 | 0.1 |
| Color FDC Blue No. 2 | 0.01 | 0.01 | 0.01 |
| Hardness | | | |
| Fast Cₘₐₓ ratio % | 168 | 138 | 81 |
| Fast AUC ratio % | 150 | 136 | 106 |
| Fed Cₘₐₓ ratio % | 121 | 119 | 101 |
| Fed AUC ratio % | 122 | 106 | 103 |
| Relative food effect | 0.72 | 0.86 | 1.25 |

Absolute pharmacokinetic data:

TABLE 3

| | | SKELAXIN ® | Ratio |
|---|---|---|---|
| | Example 4 | (Es803488A) | (Rest/Ref) |
| Cₘₐₓ (fast) ng/mL | 2649.3 | 1537.6 | 168 |
| AUC (fast) ng/mL | 21113.2 | 14181.9 | 150 |
| Cₘₐₓ (Fed) ng/mL | 3765.7 | 3108.8 | 121 |
| AUC (Fed) ng/mL | 21829.1 | 17913.3 | 122 |
| Ratio | 70.35 | 49.46 | |
| Cₘₐₓ fast/Cₘₐₓ fed | | | |
| Ratio | 96.72 | 79.17 | |
| AUCₘₐₓ fast/AUCₘₐₓ fed | | | |
| | | SKELAXIN ® | Ratio |
| | Example 6 | (Es803488A) | (Rest/Ref) |
| Cₘₐₓ (fast) ng/mL | 1630.34 | 1952.3 | 81 |
| AUC (fast) ng/mL | 15855.1 | 14670.98 | 106 |
| Cₘₐₓ (Fed) ng/mL | 3208.17 | 3161.7 | 101 |
| AUC (Fed) ng/mL | 17253 | 16745.4 | 103 |
| Ratio | 50.82 | 61.75 | |
| Cₘₐₓ fast/Cₘₐₓ fed | | | |
| Ratio | 91.90 | 87.61 | |
| AUCₘₐₓ fast/AUCₘₐₓ fed | | | |

It can be seen from table 3 that the fasting Cₘₐₓ can be decreased by lowering the amount of disintegrant while not significantly lowering the fed Cₘₐₓ

While it is apparent that the invention disclosed herein is well calculated to fulfill the objects stated above, it will be appreciated that numerous modifications and embodiments may be devised by those skilled in the art. Therefore, it is intended that the appended claims cover all such modifications and embodiments as falling within the true spirit and scope of the present invention.

What is claimed:

1. A pharmaceutical composition, comprising more than 400 mg of metaxalone, at least one binder, and at least one disintegrant, wherein the weight ratio between the binder and metaxalone is in the range of about 1 percent to about 10 percent, and the weight ratio between the disintegrant and metaxalone is in the range of about 0.5 percent to about 10 percent.

2. The pharmaceutical composition according to claim 1, wherein the composition has a relative food effect of about 0.6 to about 1.2.

3. The pharmaceutical composition according to claim 1, wherein the composition has a food effect of about 1 to about 1.8.

4. The pharmaceutical composition according to claim 1, wherein the binder is a polyvinylpyrrolidone compound.

5. The pharmaceutical composition according to claim 4, wherein the binder is selected from the group consisting of Povidone K-30 and Povidone K-90.

6. The pharmaceutical composition according to claim 1, wherein the disintegrant has at least one carboxylic group.

7. The pharmaceutical composition according to claim 6, wherein the disintegrant is selected from the group consisting of croscarmellose sodium and sodium starch glycolate.

8. The pharmaceutical composition according to claim 1, wherein the disintegrant is sodium starch glycolate, and the binder is polyvinylpyrrolidone.

9. The pharmaceutical composition according to claim 1, wherein the disintegrant is sodium starch glycolate and the binder is Povidone K-90.

10. The pharmaceutical composition according to claim 1, wherein the weight ratio between the disintegrant and metaxalone is in the range of about 0.5 percent to about 3 percent.

11. The pharmaceutical composition according to claim 1, wherein the weight ratio between the binder and metaxalone is in the range of about 3 percent to about 9 percent.

12. A method for producing the pharmaceutical composition according any of claims 1 to 11, having controlled bioavailability and controlled relative food effect, the method comprising the steps of:
(a) preparing a pharmaceutical composition comprising more than 400 mg of metaxalone together with at least one binder and at least one disintegrant;
(b) calculating the food effect and/or the relative food effect of the prepared pharmaceutical metaxalone composition;
(c) adjusting the binding capacity of the binder and the disintegration capacity of the disintegrant to achieve a desired food effect and/or relative food effect; and
(d) preparing a pharmaceutical composition, comprising more than 400 mg of metaxalone together with at least one binder and at least one disintegrant, having the adjusted binding capacity and disintegration capacity, wherein the weight ratio between the binder and metaxalone is in the range of about 1 percent to about 10 percent, and the weight ratio between the disintegrant and metaxalone is in the range of about 0.5 percent to about 10 percent; and
(e) determining the food effect and/or the relative food effect of the prepared metaxalone composition.

13. The method according to claim 12, further comprising comparing the food effect and/or the relative food effect of the prepared metaxalone composition to the desired food effect and/or relative food effect; and
when the food effect and/or the relative food effect of the metaxalone composition is greater than the desired food effect and/or relative food effect, the amount and/or the strength of the binder and the disintegrant is increased, and steps (d) and (e) are repeated until a pharmaceutical metaxalone composition, having a food effect and/or relative food effect value about that of the desired food effect and/or relative food effect, is obtained.

14. The method according to claim 12, further comprising comparing the food effect and/or the relative food effect of the prepared metaxalone composition to the desired food effect and/or relative food effect; and
when the food effect and/or relative food effect of the metaxalone composition is less than the desired food effect and/or relative food effect, the amount and/or the strength of the binder and the disintegrant is decreased, and steps (d) and (e) are repeated until a pharmaceutical metaxalone composition, having a food effect and/or relative food effect value about that of the desired food effect and/or relative food effect, is obtained.

15. The method according to claim 12, wherein the relative food effect is about 0.6 to about 1.2.

16. The method according to claim 12, wherein adjusting the binding capacity of the binder and the disintegration capacity of the disintegrant comprises increasing or decreasing both the disintegration capacity of the disintegrant and the binding capacity of the binder.

17. The method according to claim 16, wherein the binding capacity and the disintegration capacity are increased.

18. The method according to claim 12, wherein adjusting the binding capacity of the binder and the disintegration capacity of the disintegrant comprises increasing or decreasing the disintegration capacity of the disintegrant.

19. A method according to claim 12, wherein adjusting the binding capacity of the binder and the disintegration capacity of the disintegrant comprises increasing or decreasing the binding capacity of the binder.

20. A method according to claim 12, wherein adjusting the binding capacity of the binder and the disintegration capacity of the disintegrant substantially increases the bioavailability values at fasted conditions with a minimal effect on a bioavailability values at fed conditions.

21. The method according to claim 12, wherein adjusting the binding capacity of the binder and the disintegration capacity of the disintegrant decreases the relative food effect at least to 0.6.

22. A pharmaceutical composition comprising more than 400 mg of metaxalone prepared according to the method of claim 12.

23. A pharmaceutical composition, comprising more than 400 mg of metaxalone, at least one binder, and at least one disintegrant, wherein the weight ratio between the binder and metaxalone is in the range of about 1 percent to about 10 percent, and the weight ratio between the disintegrant and metaxalone is in the range of about 0.5 percent to about 10 percent, wherein the composition is prepared according to the method of claim 12.
